(19) United States
(12) Patent Application Publication
(10) Pub. No.: US 2015/0094717 A1
(43) Pub. Date: Apr. 2, 2015

(54) DOME-SHAPED BIPOLAR ELECTRODE ASSEMBLY

(71) Applicant: Cook Medical Technologies LLC, Bloomington, IN (US)

(72) Inventor: Tyler McLawhorn, Winston-Salem, NC (US)

(21) Appl. No.: 14/495,544

(22) Filed: Sep. 24, 2014

Related U.S. Application Data
(60) Provisional application No. 61/883,315, filed on Sep. 27, 2013.

(51) Int. Cl. A61N 1/04

(52) U.S. Cl. CPC A61N 1/0472
USPC 606/48

(57) ABSTRACT

A bipolar endoscopic needle knife may an elongate tubular member and a dome-shaped bipolar electrode assembly coupled to a distal end of the elongate tubular member. The bipolar electrode assembly may include an active electrode portion coupled to an active path of the bipolar endoscopic needle knife, a return electrode portion coupled to a return path of the bipolar endoscopic needle knife, and an insulating electrode portion configured to insulate the active electrode portion from the return electrode portion. When integrated together, the active, return, and insulating electrode portions may form a dome-shaped outer surface of the bipolar electrode assembly.

### CROSS-REFERENCE TO RELATED APPLICATION

This application is a U.S. Non-Provisional Application, which claims the benefit of co-pending U.S. Provisional Application No. 61/883,315, filed Sep. 27, 2013. The contents of U.S. Provisional Application No. 61/883,315 are incorporated by reference in their entirety.

### TECHNICAL FIELD

The present invention relates generally to medical devices, and more particularly to a dome-shaped bipolar electrode assembly coupled to a catheter of an endoscopic needle knife.

### BACKGROUND

Endoscopic needle knives may be used to perform various electrosurgical medical procedures, such as endoscopic submucosal dissection (ESD) or endoscopic retrograde cholangiopancreatography (ERCP) on tissue within a patient. An endoscopic needle knife may be used as an alternative to a sphincterotome, particularly where the sphincterotome is unable to cannulate the papilla.

Typical endoscopic needle knives may include a cutting wire disposed within a catheter. To perform the electrosurgical procedure, the cutting wire may be distally advanced so that a distal end of the cutting wire extends past the catheter to a desired location, where the distal end of the cutting wire is then exposed to the tissue outside the catheter. The distal end of the cutting wire may contact the tissue, and an electrical current may be sent along the cutting wire to perform the electrical procedure.

For bipolar configurations, both the cutting wire and a return electrode must sufficiently contact the tissue for the electrical current to be sent along the cutting wire to the treatment site. Sufficient contact may occur when both the cutting wire and the return electrode contacts the tissue, and also when the cutting wire and return electrode are contacting the tissue such that a desired or adequate surface area contact ratio between the cutting wire and the return electrode is achieved. This ensures a proper current density ratio between the cutting wire and the return electrode when performing the electrosurgical procedure in a bipolar manner.

Due to having to move the cutting wire to perform the electrosurgical procedure, it may be difficult for a user of a typical bipolar endoscopic needle knife to position the cutting wire at a desired location outside the catheter so that both the cutting wire and the return electrode sufficiently contact the tissue, and so that the sufficient contact is maintained during performance of the electrosurgical procedure. This may be even more difficult if the distal end of the bipolar needle knife has to approach the tissue at the treatment site from a tangential or perpendicular angle.

### BRIEF SUMMARY

An aspect of the present disclosure may include a bipolar endoscopic needle knife that may include an elongate tubular member; and a bipolar electrode assembly coupled to a distal end of the elongate tubular member. The bipolar electrode assembly may include an active electrode portion, a return electrode portion, and an insulating electrode portion. The active, return, and insulating electrode portions, when integrated together, may form a dome-shaped outer surface of the bipolar electrode assembly.

Another aspect of the present disclosure may include a bipolar electrode assembly for an endoscopic needle knife. The bipolar electrode assembly may include an active electrode portion; a return electrode portion; and an insulating electrode portion. The active, return, and insulating electrode portions, when integrated together, may form a dome-shaped outer surface of the bipolar electrode assembly. Additionally, the active electrode portion may be disposed on an outer surface of the insulating electrode portion. The insulating electrode portion may electrically insulate the active electrode portion from the return electrode portion when the active electrode portion is disposed on the outer surface of the insulating electrode portion.

### BRIEF DESCRIPTION OF THE DRAWINGS

FIG. 1 is a perspective view of distal portion of a bipolar endoscopic needle knife that includes a dome-shaped bipolar electrode assembly coupled to a distal end of an elongate tubular member.

FIG. 1A is a perspective view of an alternative embodiment of a bipolar endoscopic needle knife, showing an active electrode embedded in an insulating electrode.

FIG. 2 is a partial cross-sectional side view of the bipolar endoscopic needle knife shown in FIG. 1.

FIG. 3A is a perspective view of a return electrode portion of the dome-shaped bipolar electrode assembly of FIG. 1, showing the return electrode portion in isolation.

FIG. 3B is a perspective view of an insulating electrode portion of the dome-shaped bipolar electrode assembly of FIG. 1, showing the insulating electrode portion in isolation.

FIG. 3C is a side view of an active electrode portion of the dome-shaped bipolar electrode assembly of FIG. 1, showing the active electrode portion in isolation and coupled to an active wire.

FIG. 4A is a perspective view of an alternative return electrode portion shown in isolation.

FIG. 4B is a partial cross-sectional side view of an alternative insulating electrode portion integrated with an alternative active electrode portion.

FIG. 5A is a perspective view of a second alternative return electrode portion shown in isolation.

FIG. 5B is a partial cross-sectional side view of a second alternative insulating electrode portion integrated with a second alternative active electrode portion.

FIG. 5C is a perspective view of the second alternative insulating electrode portion shown in isolation.

FIG. 6A is a perspective view of a third alternative return electrode portion shown in isolation.

FIG. 6B is a partial cross-sectional side view of a third alternative insulation electrode portion integrated with a third alternative active electrode portion.

FIG. 7A is a perspective view of a distal portion of an electrosurgical device having a dome-shaped bipolar electrode assembly being distally advanced to a treatment site.

FIG. 7B is a perspective view of the distal portion of the electrosurgical device of FIG. 7A inserted in an opening of the treatment site.

FIG. 7C is a perspective view of the distal portion of the electrosurgical device of FIGS. 7A and 7B, rotated from the position shown in FIG. 7B.

### DETAILED DESCRIPTION

The present disclosure describes a bipolar endoscopic needle knife that includes a dome-shaped bipolar electrode assembly coupled to a distal end of a catheter. The bipolar endoscopic needle knife may be configured to cut tissue at a treatment site within a patient through an electrosurgical procedure that involves transmission of electrical current to the treatment site. To transmit the electrical current, the bipolar endoscopic needle knife may form an electrical circuit with the tissue at the treatment site. In particular, the bipolar needle knife may include a pair of conductive paths, including an active path and a return path, that contacts the tissue. The tissue may function as a load for the circuit due to the tissue's inherent resistive properties. When contact is made and the electrical circuit is formed, the electrical current may be transmitted down the active path, cut a portion of the tissue as the current passes through the tissue, and return through the return path. The bipolar endoscopic needle knife may have a bipolar configuration in that the return path may be attached to, integrated with, disposed within, or included as part of the catheter.

The dome-shaped electrode assembly may include both an active electrode portion that is part of the active path and a return electrode portion that is part of the return path for the bipolar configuration. The active electrode portion and the return electrode portion may be configured to contact the tissue to form the electrical circuit. The dome-shaped electrode assembly may further include an insulating portion that is configured to electrically insulate the active electrode portion from the return electrode portion.

The active, return, and insulating electrode portions may be integrated or combined to form a dome-shaped outer surface of the bipolar electrode assembly. The dome-shaped outer surface may have a rounded, atraumatic distal tip. In addition, when the electrode portions are integrated together, the active and return electrode portions may be in fixed positions relative to each other. That is, to perform the electrosurgical procedure, the active electrode portion does not move relative to the return electrode portion. Also, the active and return electrode portions may be sized so that their respective surface areas yield at least a minimum current density ratio suitable for the bipolar configuration. Further, the active and return electrode portions may be integrated relative to each other to facilitate sufficient contact with the tissue for a variety of angles at which the electrode assembly may approach and contact the tissue.

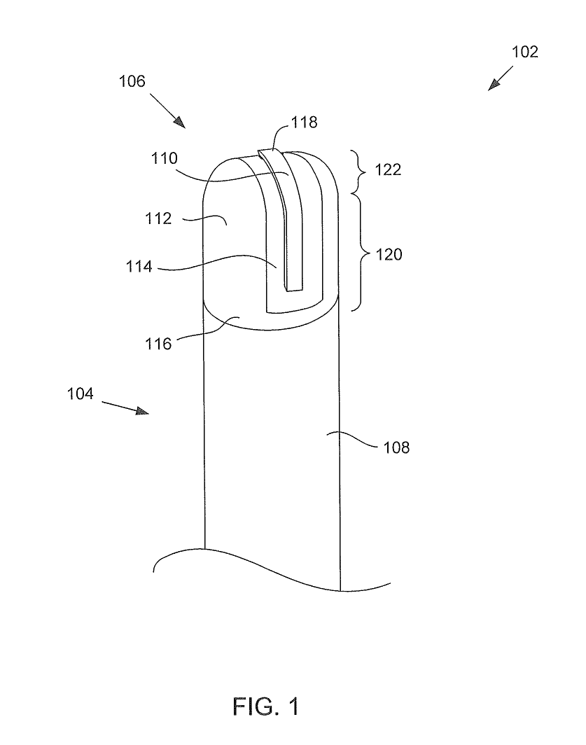

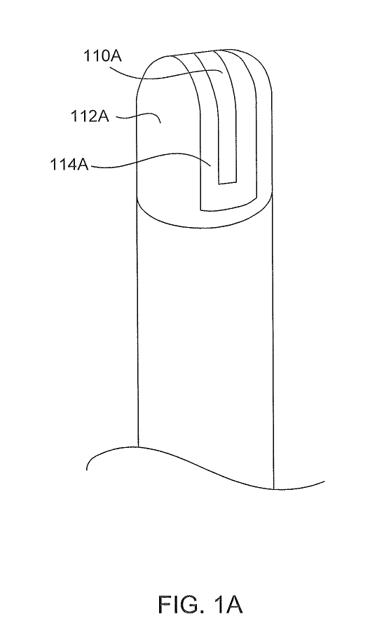

FIG. 1 shows a side view of a distal portion of a bipolar endoscopic needle knife 102 configured to cut tissue at a treatment site within a patient during an electrosurgical procedure. The bipolar endoscopic needle knife 102 may include an elongate tubular member 104 and a dome-shaped bipolar electrode assembly 106. The domed-shaped bipolar electrode assembly 106 may be coupled to a distal portion 108 of the tubular member 104.

The dome-shaped bipolar electrode assembly 106 may include an active electrode portion 110, a return electrode portion 112, and an insulating portion 114. The active electrode portion 110 may be part of an active path of the bipolar endoscopic needle knife 102 and configured to contact the tissue to electrically couple the active path to the tissue. The return electrode portion 112 may be part of a return path of the bipolar endoscopic needle knife 102 and be configured to contact the tissue to electrically couple the return path to the tissue. When both the active electrode portion 110 and the return electrode portion 112 make sufficient contact with the tissue, an electrical circuit may be formed and electrical current may be supplied from a power source (not shown) to the treatment site to cut the tissue during the electrosurgical procedure. The insulating portion 114 may electrically insulate the active electrode portion 110 from the return electrode portion 112. The insulating portion 114 may be made of an insulating material, such as PTFE or polypropylene, as examples. The insulating portion 114 may provide sufficient spacing or separation between the active electrode portion 110 and the return electrode portion 112 so that the active electrode portion 110 and the return electrode portion 112 are sufficiently insulated from each other and to prevent any shorting between the electrodes 110, 112.

When integrated or combined together, the active, return, and insulating portions 110, 112, 114 may form a bipolar electrode assembly that has a generally dome-shaped outer surface. The dome shape may be determined or defined by an outer diameter over a longitudinal length of the electrode assembly 106. In particular, the outer diameter may have a size that distally converges from an initial size at a proximal end 116 to a distal point at a distal-most end or tip 118 of the electrode assembly 106. The initial size of the outer diameter of the proximal end 116 may be the same or substantially the same as the outer diameter of tubular member 104 to which it is coupled. Example outer diameters may be in a range from 7 French (0.092 inches) to 10 French (0.131 inches), although other outer diameters may be used. As shown in FIG. 1, when the electrode assembly 106 is coupled to the tubular member 104, the outer surface of the tubular member 104 and the outer surface of the electrode assembly 106 at the proximal end 116 may be flush or substantially flush with each other.

In addition, the dome-shaped outer surface may be separated or divided into two portions, a proximal surface portion 120 and a distal surface portion 122. The outer diameter over the distal surface portion 122 may distally converge in a rounded or spherical manner such that the distal surface portion 122 is a rounded, hemispherical, and/or atraumatic surface. For some example embodiments, as shown in FIG. 1, the outer diameter over the proximal surface portion 120 may be substantially uniform such that the proximal portion 120 has a generally cylindrical outer surface. In alternative example embodiments, the outer diameter over the proximal surface portion 120 may vary to form variously-shaped outer surfaces. As an example, the outer diameter may distally taper over the proximal surface portion 120 to form a conical-shaped outer surface. As another example, the outer diameter over the proximal portion 120 may form a bulbously-shaped outer surface. Other types of outer surfaces over the proximal portion 120 may be possible.

When combined or integrated, the outer surfaces of the return electrode portion 112 and the insulating portion 114 may form a dome shaped outer surface of the electrode assembly 106, and the active electrode portion 110 may longitudinally extend over the outer surface of the insulating portion 114. As shown in FIG. 1, the return and insulating electrode portions 112, 114 may be combined or integrated so that their outer surfaces are flush or substantially flush with each other. The active electrode portion 110 may protrude from the outer surface of the insulating electrode portion 114, as shown in FIG. 1. For example, the active electrode portion may have a thickness of about 0.1 inches, and so may be raised above or protrude from the outer surface of the insulating electrode portion 114 by about 0.1 inches. FIG. 1A shows an alternative example configuration, where an active electrode portion 110A may be embedded in an insulating electrode portion 114A, such as by being disposed in a track or recessed groove of the insulating electrode portion 114A, such that outer surface of the active electrode portion 110A is flush or substantially flush with outer surfaces of the return and insulating electrode portions 112A, 114A.

Referring back to FIG. 1, the outer surfaces of each of the active, return, and insulating electrode portions 110, 112, 114 may have an associated surface area. The electrode portions 110-114 may be configured to provide a desired contact surface area ratio that yields at least a minimum current density ratio between the active and return electrode portions 110, 112. The contact surface area ratio may be a ratio of the surface area of the outer surface of the return electrode portion 112 that is in contact with the tissue at the treatment site to the surface area of the outer surface of the active electrode portion 110 that is in contact with the tissue at the treatment site. The contact surface ratio may be a desired ratio in that it may be a ratio that is desired when the active and return electrode portions 110, 112 are both in contact with tissue at the treatment site. A minimum desired contact surface area ratio may be at least about three-to-one. For some example embodiments, the electrode portions 110-114 may be configured to provide a contact surface area ratio of about ten-to-one. Because the entire outer surfaces of the active and return electrode portions 110, 112 may not be in contact with the tissue when positioned at the treatment site, the electrode portions 110-114 may be sized or configured to have a total surface area ratio of the return electrode portion 112 to the active electrode portion 110 that is greater than the desired contact surface area ratio. The total surface area ratio may be a ratio of a total surface area of the outer surface of the return electrode portion 112 to a total surface area of the outer surface of the active electrode portion 110 (i.e., not just the portions of the return and active electrode portions 112, 110 in contact with the tissue). For some example configurations, the total surface area ratio between the return electrode portion 112 and the active electrode portion 110 may be about fourteen-to-one, although other total surface area ratios may be used.

The active electrode portion 110 may be a relatively thin strip of a conductive material, such as stainless steel or tungsten as examples, that longitudinally extends in a proximal direction from the distal tip 118 over the outer surface of the insulating portion 114. The active electrode strip 110 may longitudinally extend a suitable length for performing the electrosurgical procedure. For some example configurations, the active electrode strip 110 may extend about 90 percent of a total length of the electrode assembly 106. For electrode assemblies configured for 7 French tubular members, an example length of the active electrode strip may be about 0.13 inches, although other lengths may be used. Additionally, the active electrode strip 110 may have a width of about ten one-thousandths of an inch (0.010 inches), although other widths may be used. The outer surface of the insulating portion 114 may be sized to surround the active electrode portion 110 to sufficiently electrically insulate the active electrode portion 110 from the return electrode portion 112. The return electrode portion 112 may then be a remaining portion to form the dome shape of the bipolar electrode assembly 106.

In sum, when integrated or combined, the outer surfaces of the active, return, and insulating electrode portions 110-114 may be sized or configured such that they form a dome shape, the surface area ratio of the outer surface of the return electrode portion 112 to the outer surface of the active electrode portion 110 is at least about three-to-one, and the insulating electrode portion 114 sufficiently insulates the active electrode portion 110 from the return electrode portion 112 to prevent any shorting between the active and return electrode portions 110, 112.

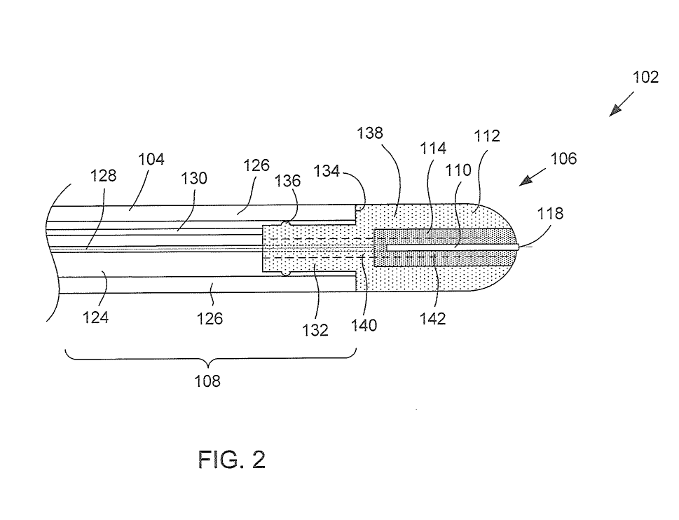

FIG. 2 shows a partial cross-sectional side view of the distal portion of the bipolar endoscope needle knife 102, with the distal portion 108 of the tubular member 104 coupled with the dome-shaped bipolar electrode assembly 106. The tubular member 104 may include a lumen 124 that longitudinally extends within a body 126 of the tubular member 104. In some example embodiments, the lumen 124 may be centrally disposed within the tubular member 104.

The bipolar endoscopic needle knife 102 may include an active wire 128 that may be disposed and longitudinally extend within the tubular member 104. The active wire 128 may be electrically coupled with the active electrode portion 110 and part of the active path of the electrical circuit configured to transmit electrical current to the treatment site to perform the electrosurgical procedure. The bipolar endoscopic needle knife 102 may also include a return wire 130 that may also be disposed and longitudinally extend within the tubular member 104. The return wire 130 may be electrically coupled with the return electrode portion 112 and part of the return path for the electrical circuit.

Each of the active and return wires 128, 130 may be made of a conductive material, such as stainless steel or tungsten as examples. In addition, the active wire 128 and the return wire 130 may each be coated or covered with an insulating material, such as a Parylene coating or polytetrafluoroethylene (PTFE) heat shrink as examples. By being coated or covered with an insulating material, the active and return wire 128, 130 may both extend within the lumen 124 electrically insulated from each other. In alternative example embodiments, the active wire 128 and/or the return wire 130 may not be coated or covered with an insulating material, and/or the active and return wires 128, 130 may be electrically insulated from each other by being disposed in separate or different lumens extending within the body 126. Alternatively, one or both of the active and return wires 128, 130 may be disposed or embedded within the body 126. Various configurations or combinations of configurations are possible.

To conduct electrical current, the active wire 128 and the return wire 130 may each be electrically coupled to a power source such as a radio frequency (RF) generator or an electrosurgical unit (ESU) (not shown) that is configured to generate and supply the electrical current. For some example bipolar endoscopic needle knives 102, the active wire 128 and the return wire 130 may be electrically coupled to the power source through a handle assembly (not shown) used by a user of the endoscope needle knife 102 to deliver and/or maneuver the distal portion to the treatment site within the patient.

The bipolar electrode assembly 106 may be connected or coupled to the distal portion 108 of the tubular member 104 in various ways. In an example embodiment, as shown in FIG. 2, the bipolar electrode assembly 106 may include a proximal coupling portion 132 configured to couple the electrode assembly 106 to the distal portion 108 of the tubular member 104. The proximal coupling portion 132 may be a cylindrical or tubular structure with an outer diameter that may be slightly less than an inner diameter of the tubular member 104 so that the proximal coupling portion 132 may be disposed within and/or inserted into the lumen 124 at a distal end 134 of the tubular member 104. The proximal coupling portion 132 may include a protruding or ribbed portion 136 that protrudes from an outer surface of the proximal coupling portion 132. The protruding portion 136 may be configured to create or form a friction or press fit with an inner wall of the body 126 defining the lumen 124 to couple the proximal coupling portion 132 with the distal portion 108 of the tubular member 104.

In some example embodiments of the bipolar electrode assembly 106, the proximal coupling portion 130 may be part of the return electrode portion 112 in that the proximal coupling portion 130 may be integral with, electrically coupled with, made of the same or similar conductive material, and/or part of the same return path as a distal portion 138 of the return electrode portion 112 disposed distal the tubular member 104 and having an outer surface used to form the dome-shaped outer surface of the bipolar electrode assembly 106 and that contacts the tissue during the electrosurgical procedure. In alternative example embodiments, the proximal coupling portion 130 may be a part of the bipolar electrode assembly 106 that is considered separate from return electrode portion 112 in that the proximal coupling portion 130 may not be electrically coupled with, made of a non-conductive material, and/or not part of the return path with the dome-shaped portion of the return electrode portion 112 disposed distal the tubular member 104 and used to contact the tissue.

In alternative example embodiments, the bipolar electrode assembly 106 may be coupled or connected to the distal portion 108 of the tubular member 108 in ways other than or in addition to using the proximal coupling portion 132. For example, an adhesive material may be used to affix the return electrode portion 112 to the body 124 of the tubular member 104 at the distal end 134. For these alternative example embodiments, the dome-shaped bipolar electrode assembly 106 may or may not include the proximal coupling portion 130. That is, some alternative example embodiments of the bipolar electrode assembly may include only the distal portion 138.

For some example embodiments, as shown in FIGS. 1 and 2, the active electrode portion 110 may distally extend over the outer surface of the insulating electrode portion 114 to the distal end 118 of the electrode assembly 106, where the active electrode portion 110 may be connected and electrically coupled to the active wire 128. As shown by dotted lines in FIG. 2, the return electrode portion 112 may include a lumen 140, and the insulating electrode portion 114 may include a lumen 142. The lumens 140, 142 may be axially aligned with each other and each extend through their respective electrode portions 112, 114. The lumen 142 of the insulating portion 114 may extend to the distal end 118, where the lumen 142 may provide an opening. The active wire 128 may distally extend through the lumens 140, 142 to the distal end 118, where the active wire 128 may be connected and electrically coupled to the active electrode portion 110. Because the active wire 128 is coated or covered with an insulating material, as previously described, the active wire 128 may extend through the lumen 140 of the return electrode portion 112 without shorting occurring between the active and return paths. Additionally, the return wire 130 may be connected and electrically coupled to the return electrode portion 112. As shown in FIG. 2, the return wire 128 may be connected to the proximal coupling portion 132, although other areas of the return electrode portion 112, such as areas on the distal portion 138 of the return electrode portion 112, may be used, particularly for embodiments of the return electrode portion 112 and/or the electrode assembly 106 generally that do not include the proximal coupling portion 132.

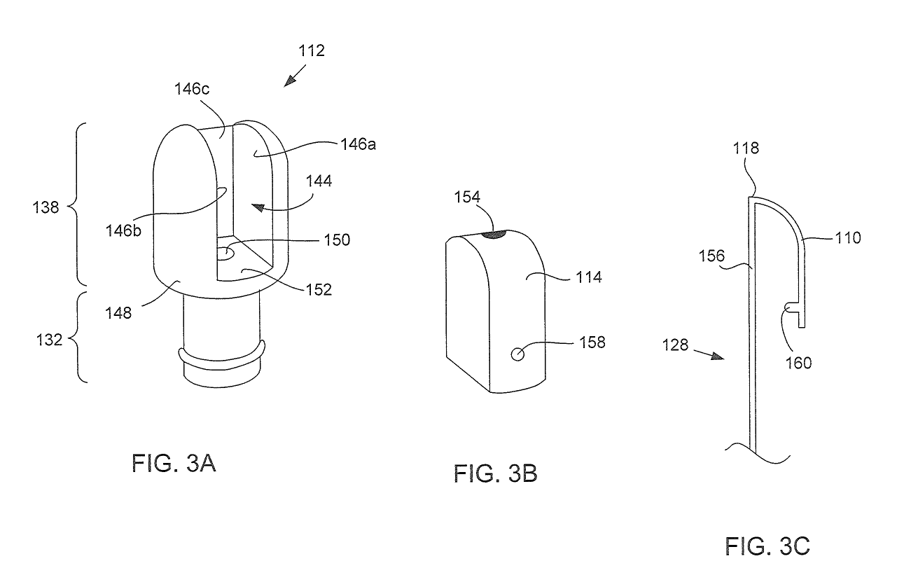

FIGS. 3A, 3B, and 3C show perspective views of the return electrode portion 112, the insulating electrode portion 114, and the active electrode portion 110, respectively, in isolation from each other. The distal portion 138 of the return electrode portion 112 may be a generally dome-shaped structure with a slot or cutout portion 144 extending in the dome-shaped structure that is configured to receive and mate with the insulating electrode portion 114. An inner side surface 146 defining in part the slot 144 may extend inwardly from the outer surface of the distal portion 138 so that when the insulating electrode portion 114 is disposed in the slot 144, the outer surface of the insulating electrode portion 114 is part of the dome-shaped outer surface of the electrode assembly 106. In the example embodiment shown in FIG. 3, the inner side surface 146 may include three surface portions having a semi-rectangular profile, with two side surface portions 146a, 146b that face each other, and a back surface portion 146c that is adjacent and oriented substantially perpendicular to each of the side surface portions 146a, 146b. Any other type profile—such as rounded, semi-circular, or polygonal as examples—may be used instead.

In addition, the distal portion 138 may include a circular or disc-shaped base portion 148. For embodiments of the dome-shaped electrode assembly 106 that include the proximal coupling portion 132, the base portion 148 may be connected to the proximal coupling portion 132. The lumen 140 may extend through the through the base portion 148. As shown in FIG. 3, the slot 144 may have a sufficient depth such that lumen 140, extending through the base portion 148, may provide an opening 150 into the slot 144 at a top surface 152 of the base portion 148.

Referring to FIG. 2B, the insulating electrode portion 114 may be sized and shaped to mate with and be disposed in the slot 144. When the insulating electrode portion 114 is disposed in the slot 144, the outer surfaces of the insulating electrode portion 144 and the distal portion 138 may be substantially flush with each other and form the dome-shaped outer surface of the bipolar electrode assembly 106. A width of the insulating electrode portion 114, and a corresponding width of the slot 144 defined by the inner side surface portions 146a, 146b may be sufficiently sized so that the active electrode portion 110 is sufficiently insulated from the return electrode portion 112 when the active electrode portion 110 is disposed on the outer surface of the insulating electrode portion 114. Additionally, the lumen 142 (FIG. 2) may distally extend to the outer surface of the insulating electrode portion 114 at the distal end 118 of the electrode assembly 106 to provide an opening 154 at the distal end 118 of the electrode assembly 106.

FIGS. 3A and 3B show the inner side surface 146 and the corresponding portions of the outer surface of the insulating electrode portion 114 to be relatively smooth and flat. Alternatively, the surfaces may include one or more grooves, notches, bumps, tracks, knobs, ribs, or other securing mechanisms that may mate or cooperate with each other to assist in securing the insulating electrode portion 114 in the slot 144. Other securing mechanisms, such as an adhesive material applied to the surfaces, may additionally or alternatively be used to secure and/or affix the insulating electrode portion 114 in the slot 144.

FIG. 3C shows a distal portion 156 of the active wire 128 connected to the active electrode portion 110. As previously described, the active electrode portion 110 may be disposed on and longitudinally extend over the outer surface of the insulating electrode portion 114, and accordingly the shape of the active electrode portion 110 may conform to the generally straight and rounded outer surface profiles of the proximal and distal surface portions 120, 122 (FIG. 1). As shown in FIG. 3C, the active electrode portion 110 may have a semi-arched or semi U-shaped profile.

In some example embodiments, the insulating and active electrode portions 114, 110 may have a cooperating securing mechanism to secure a proximal end of the active electrode portion 110 to the insulating electrode portion 114. For example, as shown in FIG. 3B and 3C, the insulating portion 114 may include a hole 158 and the active electrode portion 110 may include a knob 160 configured to mate with the hole 158. When the knob 160 is positioned in the hole 158, the proximal end of the active electrode portion 110 may be secured to the insulating electrode portion 114. Other securing mechanisms, such as adhesive material, may be used instead of or in addition to the hole 158 and knob 160.

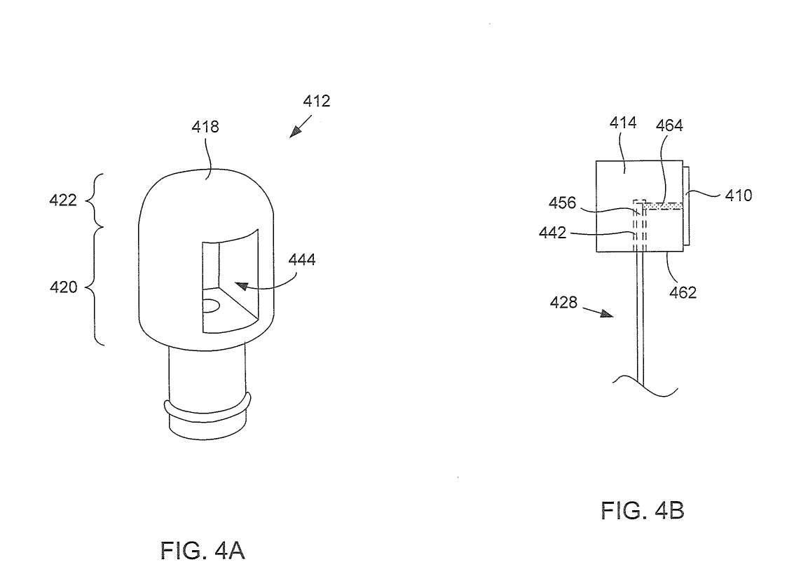

FIGS. 4A and 4B show active, return, and insulating electrode portions 410, 412, 414 of an alternative example dome-shaped bipolar electrode assembly, which may be used for the bipolar endoscopic needle knife 102 instead of the electrode assembly 106 shown in FIGS. 1, 2, and 3A-3C. FIG. 4A shows a perspective view of the return electrode portion 412 in isolation. FIG. 4B shows a partial cross-sectional side view of the insulating electrode portion 414 integrated with a distal portion of the active path.

For the alternative dome-shaped bipolar electrode assembly shown in FIGS. 4A, 4B, the active electrode portion 410 may not extend distally all the way to a distal end 418 of the electrode assembly, but instead may extend to a position proximal the distal end 418. For example, as shown in FIGS. 4A and 4B, the active electrode portion 410 may extend over a generally constant outer-diameter proximal surface portion 420 of the dome-shaped electrode assembly, but may not extend over the rounded distal surface portion 422. As such, as shown in FIG. 4A, all or substantially all of the rounded distal surface portion 422 may include the outer surface of the return electrode portion 412. A slot 444 extending in the return electrode portion 412 and the correspondingly sized insulating electrode portion 414 may be sized so that an outer surface of the insulating electrode portion is flush with the outer surface of the return electrode portion 412, and so that the insulating electrode portion 414 sufficiently separates and insulates the active electrode portion 410 from the return electrode portion 412.

Referring to FIG. 4B, a lumen 442 may longitudinally extend at least partially through the insulating electrode portion 414 from a proximal end 462. A distal portion 456 of an active wire 428 may extend in the lumen 442. A conductive member or coupling segment 464 may be included to electrically couple the distal portion 456 of the active wire 428 with the active electrode portion 410. As shown in FIG. 4B, the conductive coupling segment 464 may transversely extend from the lumen 442 to the outer surface of the insulating electrode portion 414. The conductive coupling segment 464 may have various configurations. For example, the segment 464 may be a conductive wire embedded in or extending within a hole or bore of the insulating electrode portion 414. Alternatively, the coupling segment 464 may include solder, conductive ink, or other conductive material, which may be deposited in the hole or bore extending from the lumen 442 to the outer surface.

In other alternative example embodiments, the active electrode portion 410 may not extend distally all the way to the distal end 418, but may still distally extend over a part of the rounded distal surface portion 422. The return and insulating electrode portions 412, 414 may be sized and shaped accordingly. In still other alternative example embodiments, the return and insulating electrode portions 112, 114 shown in FIGS. 3A and 3B may be integrated with the active electrode portion 410 shown in FIG. 4B. The insulating electrode portion 114 may be modified to include a conductive coupling segment, similar to the conductive coupling segment 464, to electrically couple a distal portion of an active wire and the active electrode. The outer surface of the electrode assembly at the distal end may include the insulating electrode portion, similar to the configuration of the electrode assembly 106, even though the active electrode portion does not distally extend to the distal end of the assembly. Various configurations or combinations of configurations shown in FIGS. 1-4 are possible.

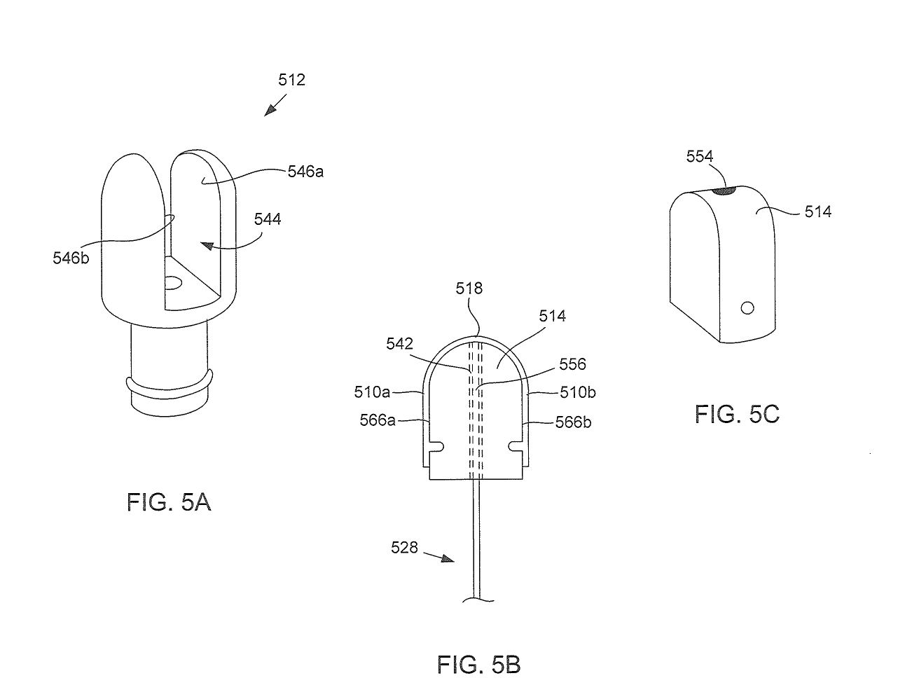

FIGS. 5A, 5B, and 5C show active, return, and insulating electrode portions 510, 512, 514 of another alternative example dome-shaped bipolar electrode assembly. FIG. 5A shows a perspective view of the return electrode portion 512 in isolation. FIG. 5B shows a partial cross-sectional side view of the insulating electrode portion 514 integrated with a distal portion of an active path. FIG. 5C shows a perspective view of the insulating electrode portion 514 in isolation. The bipolar electrode assembly shown in FIGS. 5A-5C may be similar to the bipolar electrode assembly 106 shown in FIGS. 1, 2, and 3A-3C, except that the active electrode portion 510 may include a pair of active electrode strips 510a, 510b disposed on and longitudinally extending over opposing surface portions 566a, 566b of an outer surface of the insulating portion 514 (FIG. 5B).

As shown in FIG. 5B, the pair of active electrode strips 510a, 510b may each distally extend over opposing outer surface portions 566a, 566b to a distal end 518 of the bipolar electrode assembly, where the active electrode strips 510a, 510b may be electrically coupled to each other. Disposed over the opposing outer surface portions 566a, 566b, the active electrode strips 510a, 510b may conform to the outer surface of the insulating electrode portion 514. When connected to each other, the active electrode strips 510a, 510b may have an arched or U-shaped profile. A lumen 542 may longitudinally extend through the insulating electrode portion 514. A distal portion 556 of an active wire 528 may distally extend through the lumen 542 to an opening 554 (FIG. 5C) at the distal end 518, where the distal portion 556 of the active wire 528 may be electrically coupled with the pair of active electrode strips 510a, 510b.

Referring to FIG. 5A, the bipolar electrode assembly may include a slot 544 extending in the return electrode portion 512. In comparison to the inner side surface 146 defining the slot 1344 of the return electrode portion 112 shown in FIG. 3A, an inner side surface 546 defining the slot 544 of the return electrode portion 512 may be without a back surface portion, such as the back surface portion 146c. As such, when the insulating electrode portion 514 is disposed in the slot 544, the two opposing outer surface portions 566a, 566b may both be flush with the outer surface of the return electrode portion 512, and together, the outer surfaces of the return electrode portion 512 and the insulating electrode portion 514 may form the dome-shaped outer surface of the bipolar electrode assembly. When the pair of active electrode strips 510a, 510b are disposed on the opposing outer surface portions 566a, 566b of the insulating electrode 514, both of the active electrode strips 510a, 510b may be exposed in order to contact the tissue at the treatment site.

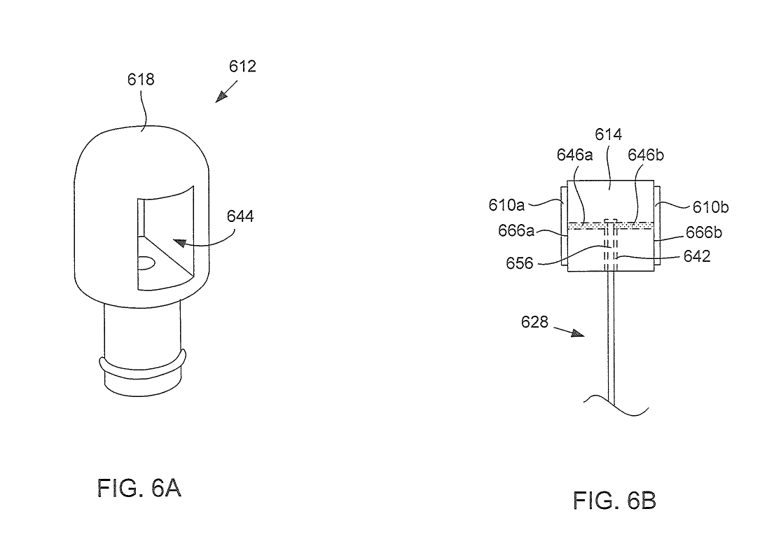

FIGS. 6A and 6B show active, return, and insulating electrode portions 610, 612, 614 of another alternative example dome-shaped bipolar electrode assembly. FIG. 6A shows a perspective view of the return electrode portion 612 in isolation. FIG. 6B shows a partial cross-sectional side view of the insulating electrode portion 614 integrated with a distal portion of an active path. The bipolar electrode assembly shown in FIGS. 6A, 6B may incorporate features of both the bipolar electrode assembly shown in FIGS. 4A, 4B and the bipolar electrode assembly shown in FIGS. 5A-5C. In particular, like the active electrode portion 510 of the bipolar electrode assembly shown in FIGS. 5A-5C, the active electrode portion 610 may include a pair of active electrode strips 610a, 610b disposed on opposing outer surface portions 666a, 666b of the insulating electrode portion 614. A slot 644 may extend in the return electrode portion 612 and configured such that when the insulating electrode portion 614 is disposed in the slot 644, the opposing outer surface portions 666a, 666b may each be flush with the outer surface of the return electrode portion 612 and together, the outer surfaces of the return electrode portion 612 and the insulating electrode portion 614 may form the dome-shaped outer surface of the bipolar electrode assembly. Also, the pair of active electrode strips 610a, 610b may each be exposed along the outer surface of the insulating electrode portion 614 in order to contact the tissue at the treatment site.

In addition, like the active electrode portion 410 of the bipolar electrode assembly shown in FIGS. 4A, 4B, the active electrode strips 610a, 610b may not extend distally all the way to a distal end 618 of the electrode assembly, but instead may extend to a position proximal the distal end 618. By not extending to the distal end 618, the pair of electrode strips 610a, 610b may be electrically disconnected at the distal end 618. Similar to the bipolar electrode assembly shown in FIGS. 4A, 4B and in order to be electrically coupled to the active path, the bipolar electrode assembly shown in FIGS. 6A, 6B may include a pair of conductive coupling segments 646a, 646b to electrically couple the pair of active electrode strips 610a, 610b with a distal portion 566 of an active wire 628, which may extend in a lumen 642 of the insulating electrode portion 614.

Bipolar electrode assemblies other than those shown in FIGS. 1-6C, including those that utilize the features or combination of the features of the bipolar electrode assemblies shown in FIGS. 1-6C, may be used. As an example, the active electrode 110 shown in FIGS. 1-3C may be used with the return and insulating electrodes 512, 515 shown in FIGS. 5A-5C. Other alternative bipolar electrode assemblies may be possible.

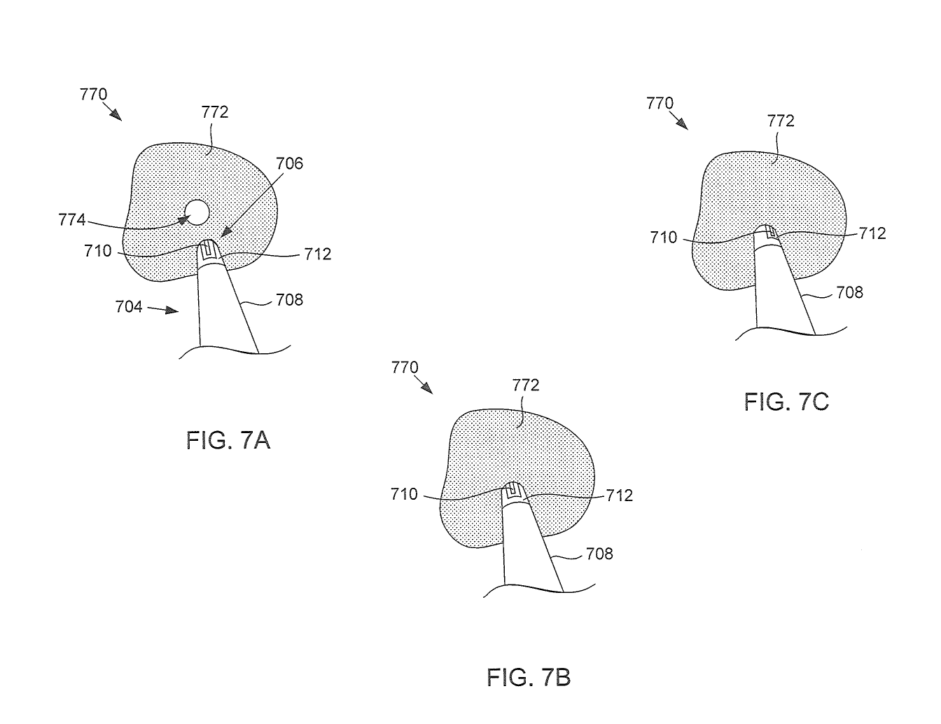

The present description also describes an example method of using an endoscopic needle knife having a dome-shaped bipolar electrode assembly to perform an electrosurgical procedure. The method is described with reference to FIGS. 7A-7C. The method may include distally advancing a dome-shaped bipolar electrode assembly 706 coupled to a distal portion 708 of an elongate tubular member 704 to a treatment site 770 located within a patient. The treatment site 770 may include an area of tissue 772 with an opening 774, such as the papilla providing an opening to the biliary tree. As shown in FIG. 7B, the bipolar electrode assembly 706 may be distally advanced such that it cannulates and is positioned in the opening 774, with an active electrode portion 710 and a return electrode portion 712 both contacting respective portions or areas of the tissue 772 surrounding the opening 774. In particular, the active and return electrode portions 710, 712 may be contacting respective portions of the tissue such that a desired contact surface area ratio between the active and return electrode portions 710, 712 may be achieved. Additionally, the bipolar electrode assembly 706 may be positioned in the opening 774 so that the active electrode portion 710 is contacting the portion of the tissue intended to be cut through performance of the electrosurgical procedure. For some example methods, the bipolar electrode assembly 706, along with the tubular member 704, may be rotated (clockwise or counter clockwise) in order for the active electrode portion 710 to contact the intended portion of the tissue to be cut, as shown in FIG. 7C. After the dome-shaped bipolar electrode assembly is positioned in the opening 774 as desired, a power source (not shown) may be activated, and electrical current may be sent to the active electrode portion 710 to perform the electrosurgical procedure. After passing through the tissue 772, the current may flow to the return electrode portion 712 and be returned back to the power source.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. A bipolar endoscopic needle knife comprising:
an elongate tubular member; and
a bipolar electrode assembly coupled to a distal end of the elongate tubular member,
wherein the bipolar electrode assembly comprises an active electrode portion, a return electrode portion, and an insulating electrode portion, and
wherein the active, return, and insulating electrode portions, when integrated together, form a dome-shaped outer surface of the bipolar electrode assembly.

2. The bipolar endoscopic needle knife of claim 1, wherein a total surface area ratio of an outer surface of the return electrode portion to an outer surface of the active electrode portion is greater than a minimum desired contact surface area ratio between the return and active electrode portions, the minimum desired contact surface area ratio being about three-to-one.

3. The bipolar endoscopic needle knife of claim 2, wherein the total surface area ratio is greater than ten-to-one.

4. The bipolar endoscopic needle knife of claim 1, wherein an outer surface of the return electrode portion and an outer surface of the insulating portion are substantially flush with each other.

5. The bipolar endoscopic needle knife of claim 1, wherein the active electrode portion comprises a strip of conductive material that longitudinally extends over an outer surface of the insulating electrode portion.

6. The bipolar endoscopic needle knife of claim 5, further comprising:
an active wire electrically connected with the active electrode portion.

7. The bipolar endoscopic needle knife of claim 6, wherein the active electrode portion distally extends to a distal end of the bipolar electrode assembly, and wherein the active electrode portion is electrically connected with the active wire at the distal end of the bipolar electrode assembly.

8. The bipolar endoscopic needle knife of claim 7, wherein each of the return electrode portion and the insulating electrode portion comprise lumens that are axially aligned with each other, and wherein the active wire extends through the axially aligned lumens to be electrically connected to the active electrode portion at the distal end of the bipolar electrode assembly.

9. The bipolar endoscopic needle knife of claim 6, wherein the active wire distally extends within the insulating electrode portion to a position proximal a distal end of the bipolar electrode assembly, and wherein a conductive member extending from within the insulating electrode portion to an outer surface of the insulating electrode portion electrically connects the active wire with the active electrode portion.

10. The bipolar endoscopic needle knife of claim 6, further comprising a return wire electrically connected with the return electrode portion, wherein the return wire and the active wire longitudinally extend within the elongate tubular member.

11. The bipolar endoscopic needle knife of claim 10, wherein the elongate tubular member comprises a body and a lumen longitudinally extending within the body, and
wherein the active wire and the return both longitudinally extend within the lumen of the tubular member.

12. The bipolar endoscopic needle knife of claim 5, wherein the strip of conductive material comprises a first strip, the active electrode portion further comprising a second strip of conductive material, wherein the first strip and the second strip longitudinally extend over opposing outer surface portions of the outer surface of the insulating electrode portion

13. The bipolar endoscopic needle knife of claim 12, wherein the first strip and the second strip are electrically connected to each other at a distal end of the bipolar electrode assembly.

14. The bipolar endoscopic needle knife of claim 5, wherein the active electrode portion protrudes from the outer surface of the insulating electrode portion.

15. The bipolar endoscopic needle knife of claim 5, wherein the active electrode portion is embedded in the insulating electrode portion such that an outer surface of the active electrode portion and the outer surface of the insulating electrode portion are substantially flush with each other.

16. The bipolar endoscopic needle knife of claim 1, wherein the bipolar electrode assembly further comprises a proximal coupling portion configured to couple the bipolar electrode assembly to the distal end of the tubular member through a press fit with the distal end of the tubular member.

17. The bipolar endoscopic needle knife of claim 16, wherein the proximal coupling portion is part of the return electrode portion.

18. The bipolar endoscopic needle knife of claim 1, wherein the return electrode portion comprises a dome-shaped structure with a slot extending in the dome-shaped structure that is configured to receive and mate with the insulating electrode portion.

19. A bipolar electrode assembly for an endoscopic needle knife, the bipolar electrode assembly comprising:
an active electrode portion;
a return electrode portion; and
an insulating electrode portion,
wherein the active, return, and insulating electrode portions, when integrated together, form a dome-shaped outer surface of the bipolar electrode assembly, and
wherein the active electrode portion is disposed on an outer surface of the insulating electrode portion, the insulating electrode portion electrically insulating the active electrode portion from the return electrode portion when the active electrode portion is disposed on the outer surface of the insulating electrode portion.

20. The bipolar electrode assembly of claim 19, wherein a total surface area ratio of an outer surface of the return electrode portion to an outer surface of the active electrode portion is greater than a minimum desired contact surface area ratio between the return and active electrode portions, the minimum desired contact surface area ratio being about three-to-one.

21. The bipolar electrode assembly of claim 20, wherein the total surface area ratio is at least ten-to-one.

22. The bipolar electrode assembly of claim 19, wherein the active electrode portion comprises a strip of conductive material that longitudinally extends over the outer surface of the insulating electrode portion.

23. The bipolar electrode assembly of claim 22, wherein the strip of conductive material comprises a first strip, the active electrode portion further comprising a second strip of conductive material, wherein the first strip and the second strip longitudinally extend over opposing outer surface portions of the outer surface of the insulating electrode portion.

24. The bipolar electrode assembly of claim 23, wherein the first strip and the second strip are electrically connected to each other at a distal end of the bipolar electrode assembly.

25. The bipolar electrode assembly of claim 19, wherein the active electrode portion protrudes from the outer surface of the insulating electrode portion.

26. The bipolar electrode assembly of claim 19, wherein the active electrode portion is embedded in the insulating electrode portion such that an outer surface of the active electrode portion and the outer surface of the insulating electrode portion are substantially flush with each other.

27. The bipolar electrode assembly of claim 19, further comprising a proximal coupling portion configured to couple the bipolar electrode assembly to the distal end of the tubular member through a press fit with a distal end of an elongate tubular member.

28. The bipolar electrode assembly of claim 27, wherein the proximal coupling portion is part of the return electrode portion.

29. The bipolar electrode assembly of claim 19, wherein the return electrode portion comprises a dome-shaped structure with a slot extending in the dome-shaped structure that is configured to receive and mate with the insulating electrode portion.

30. The bipolar electrode assembly of claim 29, wherein an outer surface of the return electrode portion and the outer surface of the insulating portion are substantially flush with each other when the insulating electrode portion is disposed in the slot extending in the dome-shaped structure.
